(19) United States
(12) Patent Application Publication
(10) Pub. No.: US 2009/0068258 A1
(43) Pub. Date: Mar. 12, 2009

(54) Caveolin-mimetic peptide for prevention and treatment of pulmonary hypertension and right ventricular hypertrophy

(76) Inventors: Michael P. Lisanti, Philadelphia, PA (US); Jean-Francois Jasmin, Philadelphia, PA (US)

Correspondence Address: AMSTER, ROTHSTEIN & EBENSTEIN LLP, 90 PARK AVENUE, NEW YORK, NY, 10016, US

(21) Appl. No.: 12/228,624

(22) Filed: Aug. 14, 2008

Related U.S. Application Data
(60) Provisional application No. 60/966,487, filed on Aug. 28, 2007.

(51) Int. Cl. A61K 9/127; A61K 38/10; A61K 38/17; C07K 7/08

(52) U.S. Cl. 424/450; 514/13; 514/12; 530/326; 530/324

(57) ABSTRACT

Methods are provided for preventing and treating pulmonary hypertension and right ventricular hypertrophy involving administering to a patient a caveolin peptide coupled to a cell permeating compound such as a cell permeating peptide.

### CROSS-REFERENCE TO RELATED APPLICATION

This application claims the benefit of U.S. Provisional Patent Application No. 60/966,487, filed Aug. 28, 2007, the content of which is incorporated by reference.

### FIELD OF THE INVENTION

The present invention relates to methods of preventing and treating pulmonary hypertension and right ventricular hypertrophy involving administering to a patient a caveolin peptide coupled to a cell permeating compound such as a cell permeating peptide.

### BACKGROUND OF THE INVENTION

Various publications are referred to throughout this application. Full citations for these references may be found at the end of the specification immediately preceding the claims. The disclosures of these publications are hereby incorporated by reference in their entireties into the subject application to more fully describe the art to which the subject application pertains.

Pulmonary hypertension (PH) includes primary pulmonary hypertension, which is inherited or occurs for unknown reasons, and secondary pulmonary hypertension, which occurs because of another condition such as heart attack, collagen vascular disease, congenital systemic to pulmonary shunts, portal hypertension, HIV infection, PH of the newborn, and intake of drugs or toxins (anorexigens). About 300 cases of primary PH are diagnosed in the United States each year while secondary PH is much more common. About 90% of heart attack patients develop PH. Despite available treatments, patients with PH still show poor prognosis. Patients with primary PH usually survive less than 3 years after the diagnosis. The survival rates of patients with secondary PH may be poorer than those of patients with primary PH because of the weakened state of the patient due to the condition giving rise to the secondary PH.

Caveolin proteins have been implicated in the development of pulmonary hypertension and the structural remodeling of the lungs⁹⁻¹². Caveolins (Cavs) are the structural proteins that are both necessary and sufficient for the formation of caveolae^{7;8}, which are vesicular organelles that are abundant in cells of the cardiopulmonary system, including endothelial cells, smooth muscle cells, epithelial cells, fibroblasts and cardiomyocytes¹⁻³. In these cell types, caveolae function in protein trafficking, cholesterol homeostasis, and signal transduction⁴⁻⁶. Cav-1 and Cav-2 are co-expressed in most cell types, while the expression of Cav-3 is muscle-specific¹⁻³. Therefore, endothelial cells, epithelial cells and fibroblasts are rich in Cav-1 and Cav-2, whereas cardiomyocytes express Cav-3¹⁻³. On the other hand, smooth muscle cells express all three caveolins^{1;2}.

Cav-1 and Cav-2 deficient mice (Cav-1^{(-/-)} and Cav-2^{(-/-)}) show abnormalities in pulmonary structure and function as demonstrated by hypercellularity, interstitial fibrosis, thickening of the alveolar septa, and reduced exercise tolerance^{9;10}. Cav-1^{(-/-)} mice were further shown to develop pulmonary hypertension and right ventricular (RV) hypertrophy¹². A marked decrease of both Cav-1 and Cav-2 protein levels has been demonstrated in the lungs of rats with myocardial infarction (MI)-induced PH¹¹. This decreased expression of pulmonary caveolins was associated with increased tyrosine-phosphorylation of the signal transducer and activator of transcription-3 (STAT3), as well as an upregulation of cyclin D1 and D3 protein levels¹¹. A reduction of pulmonary Cav-1 expression was later reported in rats with monocrotaline (MCT)- and 3-[(2,4 dimethylpyrrol-5-yl)methylidenyl]-indolin-2-one (SU5419)-induced PH^{13;14}. Decreases in both Cav-1 and Cav-2 protein levels were also recently demonstrated in plexiform lesions of patients with severe PH¹⁴. As previously suggested¹¹, down-modulation of pulmonary caveolin protein expression could thus represent an initiating mechanism leading to the development of PH and lung remodeling.

The coupling of molecules to a 16 amino acids peptide corresponding to the homeodomain of the Drosophila transcription factor antennapedia (AP or penetratin) has been shown to facilitate their uptake into cultured mammalian cells through a non-endocytic and non-degradative pathway^{15;16}. Coupling of the Cav-1 scaffolding domain to the AP peptide (AP-Cav or Cavtratin¹⁷) was recently shown to facilitate its translocation across the cell membranes and to reduce inflammation, microvascular hyper-permeability and tumor progression in mice^{17;18}. Perfusion of a Cav-1 peptide was shown to exert cardioprotective effects in myocardial ischemia-reperfusion experiments¹⁹. In contrast, it has been reported that increased smooth muscle expression of caveolin-1 and caveolae contribute to the pathophysiology of idiopathetic pulmonary arterial hypertension³⁷. Accordingly, whether in vivo modulation of caveolin protein levels could prevent the development of pulmonary hypertension and right ventricular hypertrophy has been unknown.

### SUMMARY OF THE INVENTION

The present invention provides methods of preventing and/or treating pulmonary hypertension and/or right ventricular hypertrophy involving administering to a patient a caveolin peptide coupled to a cell permeating compound such as a cell permeating peptide.

### BRIEF DESCRIPTION OF THE DRAWINGS

FIG. 1A-1B. MCT and MCT+AP rats developed PH and RV hypertrophy as shown by increases in the RV systolic pressures (A) and the RV/LV+septum weight ratio (B), respectively. Administration of AP-Cav to MCT rats significantly reduced both RV systolic pressures (A) and RV/LV+septum weight ratio (B). *p<0.01 vs control, †P<0.01 vs MCT and ‡p<0.01 vs MCT+AP (n=10 to 25 for each group).

FIG. 2A-2C. Western blot analyses of MCT and MCT+AP rat lungs show decreased expression of Cav-1 and Cav-2, as compared to Control rat lungs (A). Administration of AP-Cav to MCT rats restored the protein levels of both Cav-1 and Cav-2 (A) (three rats are shown for each group). Quantitation of Cav-1 and Cav-2 expression are shown in panels (B) and (C), respectively. Immunoblotting with β-actin is shown as a control for equal protein loading. *p<0.05 vs control, †p<0.05 vs MCT and ‡p<0.05 vs MCT+AP (n=10 to 25 for each group).

FIG. 3A-3L. Dual-label immunofluorescence analysis of Cav-1 (A,D,G,J) and vWF (B,E,H,K) expression shows a marked decrease in Cav-1 expression in the pulmonary arteries of MCT (D,E,F) and MCT+AP (G,H,I) rats as compared to Control rats (A,B,C). Administration of AP-Cav to MCT rats prevented the reduction of Cav-1 in pulmonary arteries (J,K,L). Panels (C,F,I,L) represent the merged images of Cav-1 and vWF. All pictures were taken at the same magnification of 40× and are representative of 15 fields per animal (n=10 to 25 for each group).

FIG. 4A-4L. Dual-label immunofluorescence analysis of Cav-2 (A,D,G,J) and vWF (B,E,H,K) expression shows a marked decrease in Cav-2 expression in the pulmonary arteries of MCT (D,E,F) and MCT+AP (G,H,I) rats as compared to Control rats (A,B,C). Administration of AP-Cav to MCT rats prevented the reduction of Cav-2 in pulmonary arteries (J,K,L). Panels (C,F,I,L) represent the merged images of Cav-2 and vWF. All pictures were taken at the same magnification of 40× and are representative of 15 fields per animal (n=10 to 25 for each group).

FIG. 5A-5B. Western blot analysis shows similar eNOS protein levels among the different groups (A) (three rats are shown for each group). Quantitation is shown in panel (B) (n=10 to 25 for each group). Immunoblotting with β-actin is shown as a control for equal protein loading.

FIG. 6A-6B. Western blot analysis of MCT and MCT+AP rat lungs shows increased expression of PY-STAT3 as compared to Control rat lungs (A). Administration of AP-Cav to MCT rats prevented the hyper-activation of the STAT3 signaling cascade (A) (three rats are shown for each group). Quantitation is shown in panel (B). Immunoblotting with total STAT3 is shown as a control for equal protein loading. *p<0.05 vs control, †p<0.05 vs MCT and ‡p<0.05 vs MCT+AP (n=10 to 25 for each group).

FIG. 7A-7C. Western blot analysis of MCT and MCT+AP rat lungs shows increased expression of cyclin D1 and cyclin D3 as compared to Control rat lungs (A). Administration of AP-Cav to MCT rats decreased the protein levels of both cyclin D1 and cyclin D3 (A) (three rats are shown for each group). Quantitation of cyclin D1 and cyclin D3 expression is shown in panels (B) and (C), respectively. Immunoblotting with β-actin is shown as a control for equal protein loading. *p<0.05 vs control, †p<0.05 vs MCT and ‡p<0.05 vs MCT+AP (n=10 to 25 for each group).

FIG. 8A-8E. Representative pictures of pulmonary arteries (100-150 μm) show increased percent medial wall thickness in MCT (B) and MCT+AP rats (C) as compared to Control rats (A). Administration of AP-Cav to MCT rats significantly decreased the percent medial wall thickness (D). Quantitation of all pulmonary arteries is shown in panel (E). *p<0.05 vs control, †p<0.05 vs MCT and ‡p<0.05 vs MCT+AP (n=10 for each group).

### DETAILED DESCRIPTION OF THE INVENTION

The invention provides a method of preventing and/or treating pulmonary hypertension and/or right ventricular hypertrophy comprising administering to a patient an amount of a caveolin peptide coupled to a cell permeating compound effective to prevent and/or treat pulmonary hypertension and/or right ventricular hypertrophy in a patient.

The pulmonary hypertension can be primary pulmonary hypertension or secondary pulmonary hypertension.

Pulmonary hypertension and/or right ventricular hypertrophy can be prevented, for example, by administering the caveolin peptide coupled to the cell permeating compound to a patient at risk for developing pulmonary hypertension and/or right ventricular hypertrophy. Patients at risk for developing pulmonary hypertension include, for example, subjects with heart attack, collagen vascular disease, congenital systemic to pulmonary shunts, portal hypertension or HIV infection; subjects who have ingested drugs or toxins (anorexigens); and subjects with a family history of pulmonary hypertension. Patients with pulmonary hypertension are at risk for developing right ventricular hypertrophy.

As used herein, a cell permeating compound coupled to a caveolin peptide includes a cell permeating compound coupled to a caveolin peptide directly or via a linker as well as synthesized sequences of a cell permeating compound and a caveolin peptide. The order of the sequence or coupling can be either first cell permeating compound (CPC) then caveolin peptide (Cav) or first caveolin peptide then cell permeating compound, i.e. either CPC-Cav or Cav-CPC.

Preferably, the caveolin peptide is one or more of:

(SEQ ID NO:7)

DGIWKASFTTFTVTKYWFYR (Cav-1 scaffolding domain);

(SEQ ID NO:8)

DKVWICSHALFEISKYVMYK (Cav-2 scaffolding domain);

(SEQ ID NO:9)

DGVWRVSYTTFTVSKYWCYR (Cav-3 scaffolding domain);

or a peptide which both has at least 80% identity, and preferably at least 90% identity, with Cav-1, Cav-2 or Cav-3 and inhibits a GTPase, a nitric oxide (NO) synthase (NOS), and a kinase. GTPases are enzymes that bind and hydrolyze guanosine triphosphate (GTP). Kinases include serine-threonine kinases and tyrosine kinases.

More preferably, the caveolin peptide is DGIWKASFTTFTVTKYWFYR (Cav-1) (SEQ ID NO:7).

As used herein, a cell permeating compound is a compound that when coupled to a caveolin peptide permits entry of the caveolin peptide into mammalian cells. The cell permeating compound can be, for example, any one or more of a liposome, a lipid, a fatty acid, or a peptide. Various cell permeating compounds have been described, e.g.³⁸⁻⁴⁵ Cell permeating fatty acids include palmitate and myristate. For peptides, a positive charge is critical for internalization into mammalian cells.⁴³

Preferably, the cell permeating compound is a cell permeating peptide. Preferably, the cell permeating peptide is one or more of:

(SEQ ID NO:1)

RQPKIWFPNRRKPWKK;

(SEQ ID NO:2)

RQIKIWFQNRRMKWKK (penetratin);

(SEQ ID NO:3)

a polypeptide consisting of 6-15 arginines;

(SEQ ID NO:4)

GYGRKKRRQRRRG (a TAT sequence);

(SEQ ID NO:5)

YGRKKRRQRRR (a TAT sequence);

(SEQ ID NO:6)

GRKKRRQRRRPPQ (a TAT sequence);

or a cell permeating peptide having at least 80% identity, and preferably at least 90% identity, with any of said cell permeating peptides. Preferably, the cell permeating peptide having at least 80% identity with any of said cell permeating peptides comprises at least three arginines. More preferably, the cell permeating peptide is RQPKIWFPNRRKPWKK (SEQ ID NO:1).

Preferably, the peptide consisting of the cell permeating peptide coupled to the caveolin peptide is:

(SEQ ID NO:10)

RQPKIWFPNRRKPWKK-DGIWKASFTTFTVTKYWFYR
or

(SEQ ID NO:11)

DGIWKASFTTFTVTKYWFYR-RQPKIWFPNRRKPWKK.

More preferably, the peptide consisting of the cell permeating peptide coupled to the caveolin peptide is:

RQPKIWFPNRRKPWKK-DGIWKASFTTFTVTKYWFYR (SEQ ID NO:10).

The invention also provides a cell permeating peptide having the sequence RQPKIWFPNRRKPWKK (SEQ ID NO:1). The cell permeating peptide can be coupled to a caveolin peptide. The order of the coupling can be first cell permeating peptide then caveolin peptide, or first caveolin peptide then cell permeating peptide. Preferably, the caveolin peptide is one or more of:

| | |
|---|---|
| DGIWKASFTTFTVTKYWFYR (Cav-1); | (SEQ ID NO:7) |
| DKVWICSHALFEISKYVMYK (Cav-2); | (SEQ ID NO:8) |
| DGVWRVSYTTFTVSKYWCYR (Cav-3); | (SEQ ID NO:9) |

or a peptide which both has at least 80% identity with Cav-1, Cav-2 or Cav-3 and inhibits a GTPase, a NOS and a kinase. More preferably, the caveolin peptide is DGIWKASFTTFTVTKYWFYR (Cav-1) (SEQ ID NO:7). Preferably, the cell permeating peptide coupled to the caveolin peptide comprises the peptide sequence:

(SEQ ID NO:10)

RQPKIWFPNRRKPWKK-DGIWKASFTTFTVTKYWFYR
or

(SEQ ID NO:11)

DGIWKASFTTFTVTKYWFYR-RQPKIWFPNRRKPWKK.

More preferably, the cell permeating peptide coupled to the caveolin peptide comprises the peptide sequence:

(SEQ ID NO:10)

RQPKIWFPNRRKPWKK-DGIWKASFTTFTVTKYWFYR.

The cell permeating compound coupled to caveolin peptide can be administered systemically, for example, by intraperitoneal injection, intramuscular injection, subcutaneous injection or intravenous injection or by inhalation using, for example, an aerosol nebulizer. The nebulizer can produce particles of cell permeating compound coupled to the caveolin peptide of 2.5 μm or less.

The present invention is illustrated in the following Experimental Details section, which is set forth to aid in the understanding of the invention, and should not be construed to limit in any way the scope of the invention as defined in the claims that follow thereafter.

### EXPERIMENTAL DETAILS

### Materials and Methods

Materials. Biotinylated peptides corresponding to AP ((biotin)-RQPKIWFPNRRKPWKK (SEQ ID NO:1)—(OH)) and AP-Cav ((biotin)-RQPKTWFPNRRKPWKK-DGIWKASFTTFTVTKYWFYR (SEQ ID NO:10)—(OH)) were custom synthesized (at the Tufts University Core Facility). MCT and a mouse monoclonal antibody (mAb) to β-actin were purchased from Sigma-Aldrich (St-Louis, Mo.). Cav-1 and -2 mAbs were the generous gifts of Dr. Roberto Campos-Gonzalez (BD-Pharmingen, San Diego, Calif.). A rabbit polyclonal antibody (pAb) to von Willebrand Factor (vWF), a mouse mAb to STAT3, a mouse mAb to phospho-tyrosine (PY)-STAT3, a mouse mAb to endothelial nitric oxide synthase (eNOS), as well as rabbit and mouse horseradish peroxidase (HRP)-conjugated secondary antibodies were all purchased from BD-Pharmingen. Mouse mAbs to cyclin D1 and cyclin D3 were purchased from Santa Cruz Biotechnology (Santa Cruz, Calif.). Rabbit and mouse fluorescein (FITC) and rhodamine (TRITC)-conjugated secondary antibodies were purchased from Jackson ImmunoResearch (West Grove, Pa.).

Animal Studies. This study was conducted according to the guidelines of the National Institute of Health and the Thomas Jefferson University Institute for Animal Studies.

Male Sprague-Dawley rats (Charles River, Wilmington, Mass.) weighing between 250-300 g received a single intraperitoneal (ip) injection of either 0.5 ml 0.9% NaCl or 0.5 ml 60 mg/kg MCT. Thirty minutes later, rats were randomly assigned to receive a daily ip injection of either 0.5 ml 0.9% NaCl, 0.5 ml AP (2.5 mg/kg/d) or 0.5 ml AP-Cav (2.5 mg/kg/d) for 2 weeks. This resulted in the following five groups: Control (n=17), Control+AP-Cav (n=10), MCT (n=25), MCT+AP (n=19), and MCT+AP-Cav (n=24).

At two weeks, rats were anesthetized with xylazine (10 mg/kg)-ketamine (50 mg/kg) followed by 2000 U heparin (Sigma-Aldrich). The right jugular vein and carotid artery were then isolated and incised, and millar catheters (SPR-249, Millar Instruments, Houston, Tex.) were advanced into the right and left ventricles (RV and LV) for hemodynamic measurements. The RV and LV pressures were recorded with the Ponemah P3-Data acquisition system (LDS Test and Measurement, Middleton, Wis.).

Afterwards, the lower lobe of the right lung and the heart were dissected and weighed to respectively determine pulmonary edema and RV hypertrophy, as previously described¹¹. The remaining lobes of the right lung were submerged in liquid nitrogen and frozen at −80° C. The left pulmonary artery was then cannulated and perfused with 4% paraformaldehyde (PFA) for 2 min. This was followed by perfusion of the airways with 4% PFA for 2 min. The left lung was then immersed in 4% PFA for 24 hours.

Immunoblot Analysis. Lung samples from Control (n=17), Control+AP-Cav (n=10), MCT (n=25), MCT+AP (n=19), and MCT+AP-Cav (n=24) groups were homogenized in a RIPA lysis buffer containing protease and phosphatase inhibitors. Proteins were then separated by SDS-PAGE (12% acrylamide) and transferred to nitrocellulose membranes. The membranes were placed in blocking solution for 30 min. Afterwards, the membranes were washed and incubated with a given primary antibody for 1 hour (Cav-1, -2 and β-actin) or 3 hours (eNOS, STAT3, PY-STAT3, cyclin D1, and cyclin D3). Finally, HRP-conjugated secondary antibodies were used to detect bound primary antibody using the SuperSignal chemiluminescence substrate (Pierce Biotechnology, Rockford, Ill.). Western blots for Cav-1, Cav-2, eNOS, PY-STAT3, cyclin D1 and cyclin D3 were subsequently quantitated using the NIH Image J software (using the mean gray value for each band).

Immunofluorescence Analysis. As mentioned above, the left lung of Control (n=17), Control+AP-Cav (n=10), MCT (n=25), MCT+AP (n=19), and MCT+AP-Cav (n=24) rats was dissected and perfused-fixed. Transverse sections were obtained and embedded with paraffin. Sections of 10 μm were cut and stained with hematoxylin and eosin (H&E).

Paraffin from 10 μm-thick sections was removed by immersion in xylene. These sections were then rehydrated with graded alcohol to water and blocked overnight. The sections were subsequently incubated with a given primary antibody for three hours. FITC- and TRITC-conjugated secondary antibodies were then added to the sections after a 15 min wash in PBS. After 1 hr of incubation with the secondary antibodies, the sections were washed in PBS and mounted using Prolong Gold antifade reagent (Molecular Probes, Carlsbad, Calif.).

Lung Vascular Morphometry. H&E sections of the left lung (n=10 for each groups) were microscopically assessed for the medial wall thickness of pulmonary arteries. Measurements of the luminal diameter and the medial thickness on either side were obtained using the Image J software. Measurements were made on 30 muscular arteries (<50 μm, 51-100 μm, and 100-150 μm of external diameter) per lung section. The medial wall thickness was then related to the external diameter and expressed as percent wall thickness, as previously described²⁰.

Statistical Analysis. Hemodynamic and morphometric variables as well as the mean gray value of each western blot are expressed as mean±S.E.M. Differences between the 5 groups were evaluated by ANOVA, followed by Tukey's multiple-group comparisons test. Statistical significance was assumed at p<0.05.

### Results

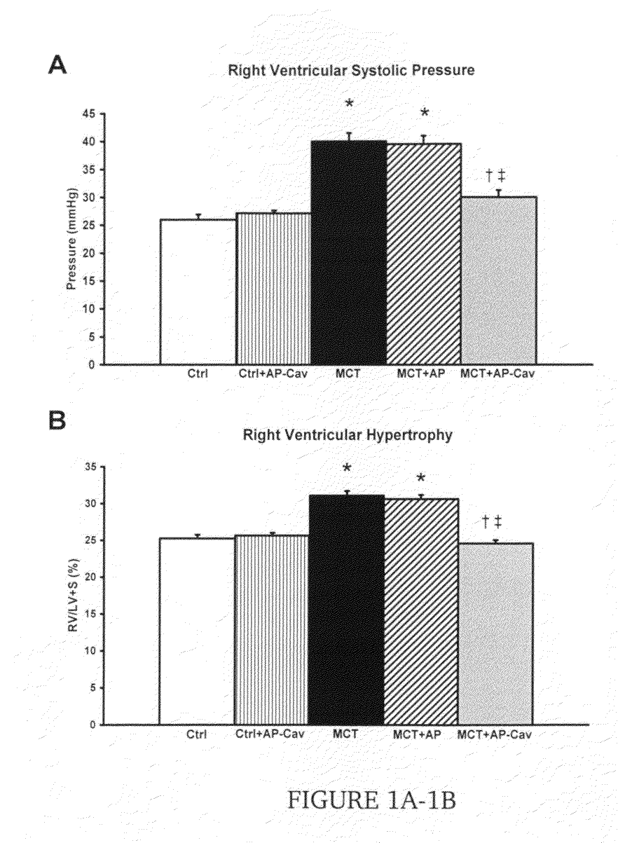

Hemodynamic and Morphological Effects of AP-Cav. The MCT and MCT+AP rats developed PH with respective RV systolic pressures of 40.2±1.5 mmHg and 39.6±1.5 mmHg, compared to 26.0±0.9 mmHg in the Control rats (p<0.01, FIG. 1A). Interestingly, the MCT+AP-Cav rats demonstrated a significant reduction of the RV systolic pressures to 30.1±1.3 mmHg (p<0.01 vs MCT and MCT+AP; FIG. 1A). The central venous pressures and RV end-diastolic pressures behaved similarly with significant increases in both MCT and MCT+AP rats, which were normalized in the MCT+AP-Cav rats (p<0.05; Table 1). The MCT and MCT+AP rats also developed RV hypertrophy with respective RV/LV+septum weight ratios of 31.1±0.6% and 30.6±0.6%, compared to 25.3±0.5% in the Control rats (p<0.01; FIG. 1B). Interestingly, the RV/LV+septum weight ratio was normalized to 24.6±0.5% in the MCT+AP-Cav rats (p<0.01; FIG. 1B). The lung weights of the MCT and MCT+AP rats were significantly increased in the absence of significant edema formation (p<0.05; Table 1). Administration of AP-Cav to MCT rats significantly reduced the lung weight (p<0.05; Table 1). Left ventricular function remained unchanged in all the experimental groups (Table 1). All other hemodynamic and morphometric variables are summarized in Table 1. Administration of AP-Cav to the Control group did not affect any of the hemodynamic and morphometric variables, as compared to the Control group receiving 0.9% NaCl alone (Table 1).

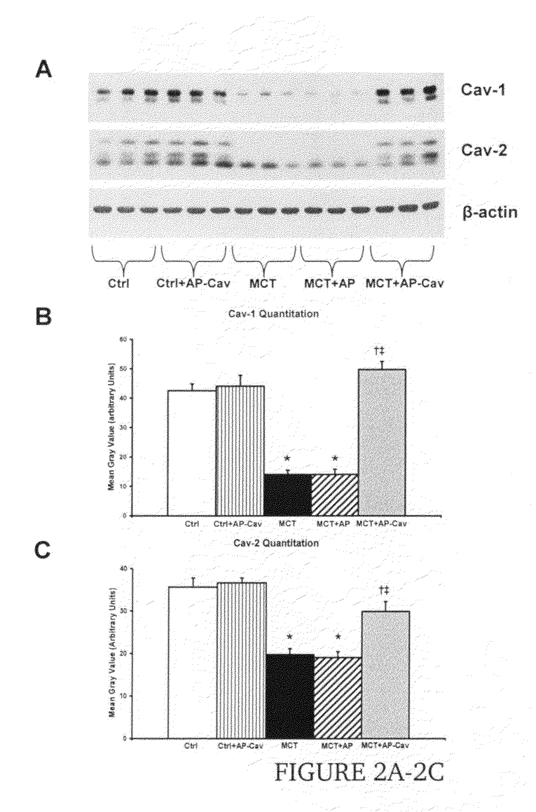

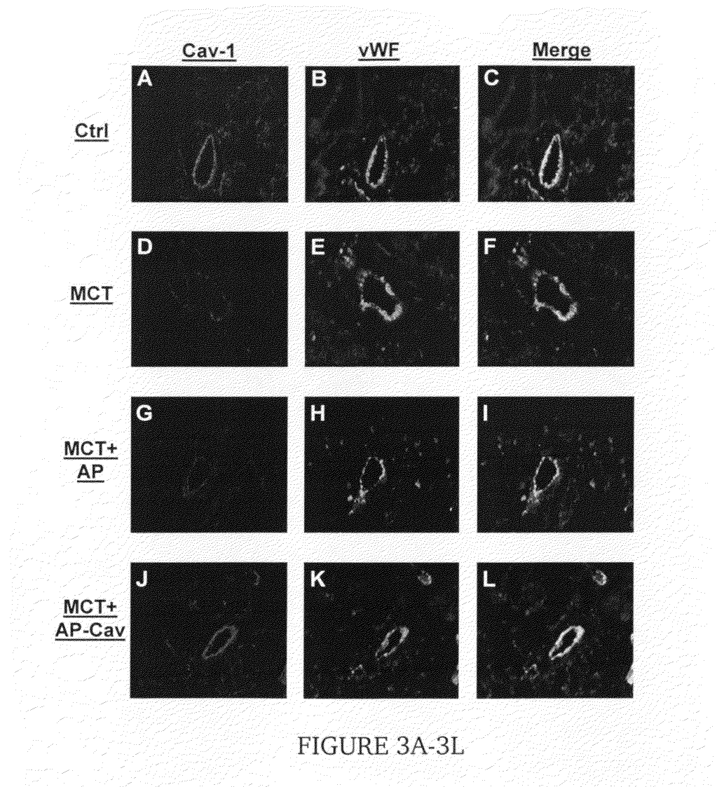

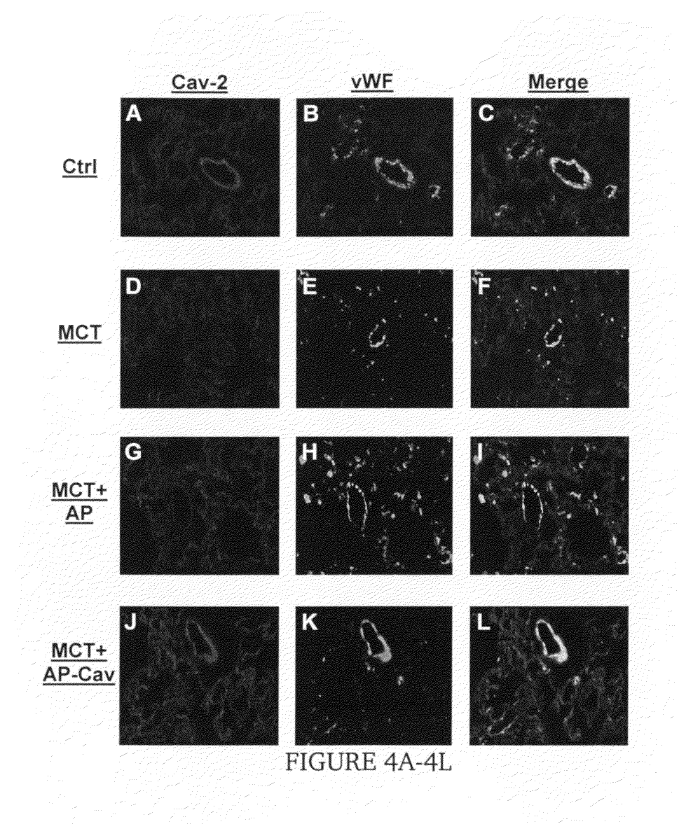

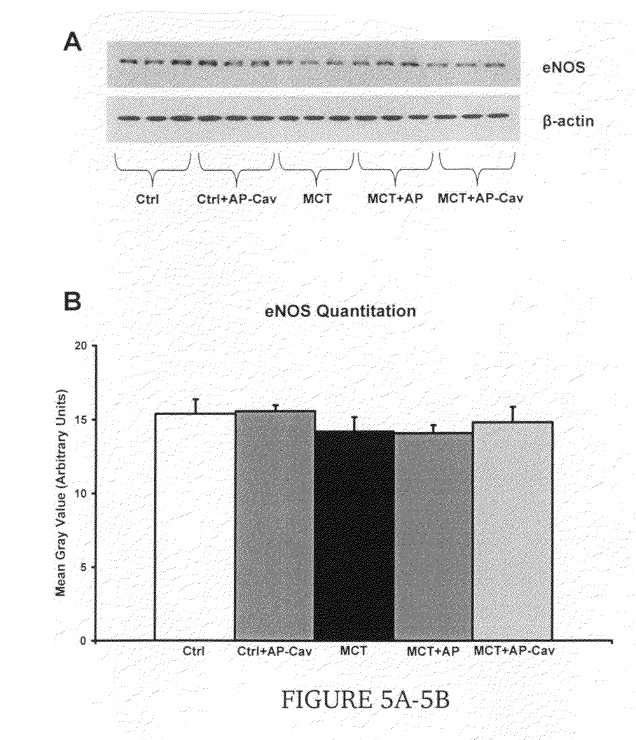

Expression of Caveolin Proteins and eNOS in the Lungs of Pulmonary Hypertensive Rats. As demonstrated in FIG. 2, immunoblot analysis showed a marked decrease of Cav-1 (˜3 fold; p<0.01) and Cav-2 (˜2 fold; p<0.01) protein levels in the lungs of MCT and MCT+AP rats. Administration of AP-Cav to MCT rats significantly prevented the reduction of pulmonary Cav-1 and Cav-2 protein levels (p<0.05; FIG. 2). Similarly, dual-label immunofluorescence analysis demonstrated marked reductions of both Cav-1 (FIG. 3) and Cav-2 (FIG. 4) expression in pulmonary arteries of MCT and MCT+AP rats, which were prevented by administration of AP-Cav. Immunoblot analysis of eNOS protein levels did not reveal any significant differences among all groups (p=ns, FIG. 5).

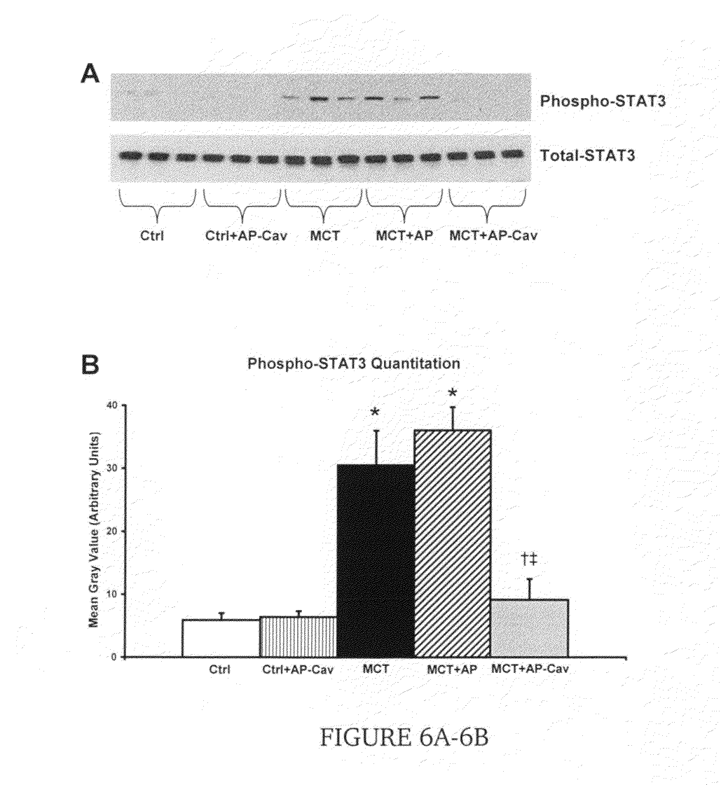

STAT3 Signaling Cascade in the Lungs of Pulmonary Hypertensive Rats. Immunoblot analysis demonstrated increased levels of PY-STAT3 (˜5 fold; p<0.05) in the lungs of MCT and MCT+AP rats (FIG. 6). Administration of AP-Cav to MCT rats prevented the hyper-activation of the STAT3 signaling cascade (FIG. 6). Importantly, the pulmonary expression of total STAT3 was similar in all groups.

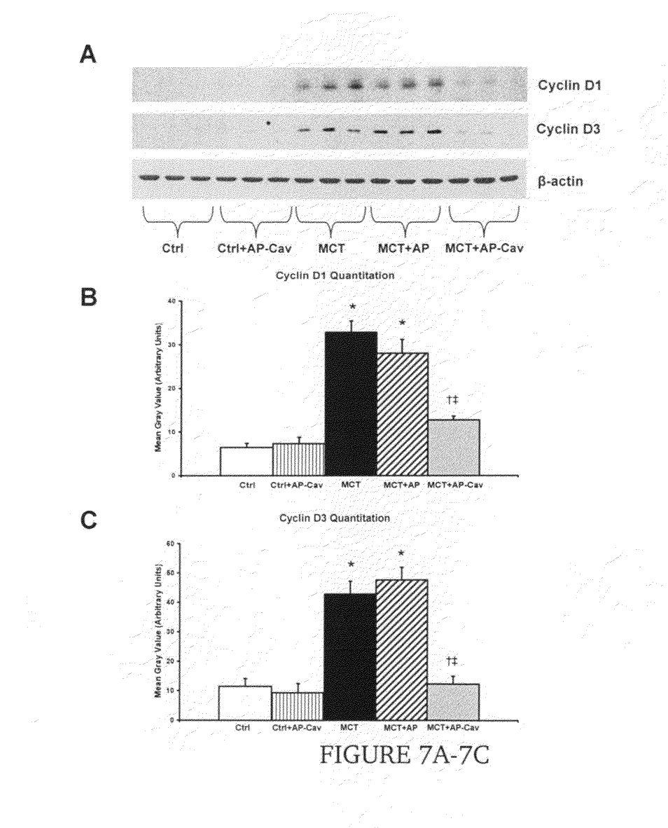

Expression of Cyclin D1 and D3 in the Lungs of Pulmonary Hypertensive Rats. As demonstrated in FIG. 7, immunoblot analysis showed marked increases in cyclin D1 (˜4 fold; p<0.01) and cyclin D3 (˜4 fold; p<0.01) protein levels in the lungs of MCT and MCT+AP rats. Interestingly, administration of AP-Cav to MCT rats prevented the upregulation of pulmonary cyclin D1 and cyclin D3 expression (p<0.01; FIG. 7).

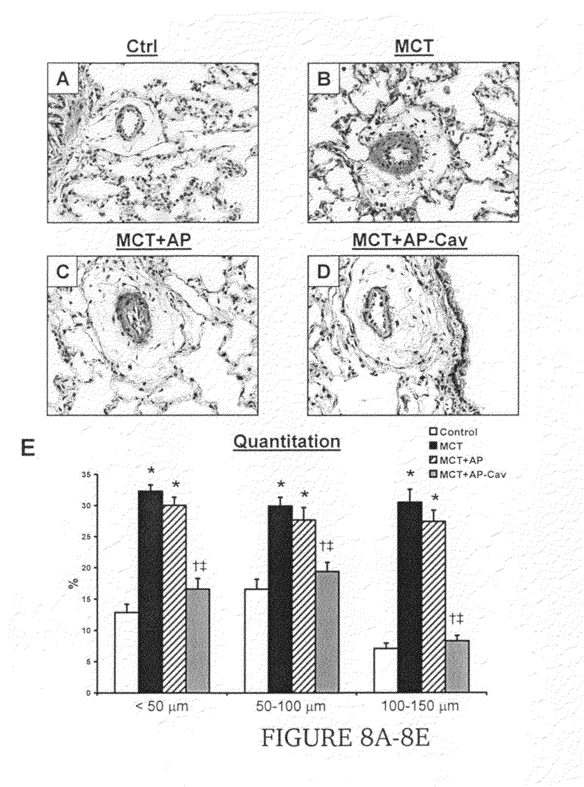

Pulmonary Arteries Remodeling. Pulmonary arteries (<50 μm, 51-100 μm, and 100-150 μm) of MCT and MCT+AP rats showed increased percent medial wall thickness (˜2-3 fold; p<0.05), as compared to Control rats (FIG. 8). Administration of AP-Cav to MCT rats significantly reduced the percent medial wall thickness (p<0.05; FIG. 8). Administration of AP-Cav to Control rats had no effect on the pulmonary arteries percent medial wall thickness (data not shown).

TABLE 1
Hemodynamic and Morphometric Variables

| Variables | Control | Control + AP-Cav | MCT | MCT + AP | MCT + AP-Cav |
|---|---|---|---|---|---|
| CVP, (mmHg) | 3.0 ± 0.2 | 3.1 ± 0.1 | 5.2 ± 0.3* | 5.1 ± 0.3* | 3.2 ± 0.2†‡ |
| RVEDP, (mmHg) | 3.1 ± 0.5 | 2.8 ± 0.2 | 5.2 ± 0.3* | 5.1 ± 0.3* | 3.0 ± 0.2†‡ |
| RV(+)dP/dt, (mmHg/s) | 1380 ± 51 | 1435 ± 60 | 1851 ± 101* | 1931 ± 78* | 1489 ± 18†‡ |
| RV(−)dP/dt, (mmHg/s) | 957 ± 36 | 988 ± 46 | 1441 ± 76* | 1395 ± 72* | 1107 ± 61†‡ |
| HR, (beats/min) | 268 ± 5 | 270 ± 6 | 274 ± 7 | 274 ± 12 | 261 ± 12 |
| MAP, (mmHg) | 81.9 ± 3.3 | 79.4 ± 3.4 | 85.8 ± 3.2 | 85.3 ± 4.4 | 82.6 ± 2.9 |
| LVEDP, (mmHg) | 3.6 ± 0.6 | 3.0 ± 0.3 | 3.6 ± 0.4 | 2.7 ± 0.3 | 2.7 ± 0.3 |
| LV(+)dP/dt, (mmHg/s) | 5905 ± 315 | 6216 ± 446 | 5965 ± 318 | 6268 ± 339 | 5766 ± 335 |
| LV(−)dP/dt, (mmHg/s) | 3958 ± 309 | 4614 ± 320 | 4345 ± 314 | 4319 ± 286 | 3891 ± 309 |
| BW (g) | 416 ± 17 | 407 ± 12 | 373 ± 12 | 335 ± 7* | 371 ± 9‡ |
| LV/BW, (%) | 0.193 ± 0.003 | 0.194 ± 0.005 | 0.195 ± 0.004 | 0.200 ± 0.004 | 0.200 ± 0.003 |
| RV/BW, (%) | 0.048 ± 0.001 | 0.050 ± 0.001 | 0.061 ± 0.002* | 0.061 ± 0.002* | 0.049 ± 0.001†‡ |
| Wet lung weight/BW (%) | 0.097 ± 0.003 | 0.091 ± 0.001 | 0.158 ± 0.007* | 0.170 ± 0.007* | 0.132 ± 0.004*†‡ |
| Dry lung weight/BW (%) | 0.021 ± 0.001 | 0.019 ± 0.000 | 0.032 ± 0.001* | 0.033 ± 0.001* | 0.028 ± 0.001*†‡ |
| Dry/wet lung weight (%) | 21.0 ± 0.4 | 20.8 ± 0.1 | 20.7 ± 0.3 | 19.7 ± 0.4 | 20.7 ± 0.3 |

CVP indicates central venous pressure; RVEDP, RV-end diastolic pressure; HR, heart rate; MAP, mean arterial pressure; LVEDP, LV-end diastolic pressure; BW, body weight.
*p < 0.05 vs Control, †p < 0.05 vs MCT and ‡p < 0.05 vs MCT + AP.

### Discussion

The present results demonstrate a marked decrease of Cav-1 and Cav-2 protein levels in the lungs of rats with MCT-induced PH. This decrease is associated with the hyper-activation of the STAT3 signaling cascade and the upregulation of cyclin D1 and cyclin D3 protein levels. Importantly, the results also demonstrate, for the first time, that short term administration of a cell-permeable Cav-1 peptide prevents the development of pulmonary artery medial hypertrophy, PH and RV hypertrophy in MCT rats. Mechanistically, administration of a Cav-1-derived peptide prevented the decreased expression of Cav-1 and Cav-2, the phosphorylation of STAT3, as well as the upregulation of cyclin D1 and cyclin D3 protein levels in the lungs of MCT rats.

Caveolin Protein and eNOS Expression in Pulmonary Hypertensive Rats. Caveolin proteins have been suggested to function as key regulators of the development of PH and lung remodeling. Indeed, the lungs of Cav-1^{(-/-)} and Cav-2^{(-/-)} mice showed hyper-cellularity, fibrosis, and thickened alveolar septa^{9;10}. Cav-1^{(-/-)} mice were further shown to develop PH and RV hypertrophy¹². Interestingly, decreased expression of pulmonary caveolins has been reported in several animal models of PH, such as the MCT, MI and SU5419 rat models^{11;13;14}. Importantly, these reports as well as the present results, appear to be relevant to human PH as decreases in both Cav-1 gene and protein expression have also been reported in patients with severe PH^{14;21}. The present results validate those of Mathew et al. (2004), which demonstrated a decrease in Cav-1 protein levels in the lungs of MCT rats. As previously reported in the MI rat model of PH¹¹, the present results further demonstrate the down-regulation of Cav-2 protein levels in the lungs of MCT rats. Moreover, the dual-label immunofluorescence analysis also validate the results of Mathew et al. (2004), which showed a reduction in Cav-1 expression in pulmonary artery endothelial cells of MCT rat lungs. Interestingly, the dual-label immunofluorescence analysis also demonstrated a reduction of Cav-2 expression in pulmonary artery endothelial cells of MCT rat lungs. Most importantly, the present studies further show that administration of AP-Cav to MCT rats prevents the reduction of pulmonary Cav-1 and Cav-2 protein levels.

Although eNOS protein levels appear slightly reduced in all MCT-treated rats, no significant differences were observed among all groups. However, previous studies reported decreased expression of pulmonary eNOS at five and six weeks post-MCT 22;23. Therefore, it is likely that significant modulations of eNOS expression appear at a later stage of the development of MCT-induced PH. Interestingly, although Cav-1 is well recognized for its negative regulation of eNOS activity²⁴, administration of AP-Cav to both Control and MCT rats did not have significant effect on pulmonary eNOS expression. The lack of effect of the AP-Cav administration on any hemodynamic variables in Control rats also supports the absence of effect of AP-Cav on eNOS activity.

STAT3 Activation and Cyclins Expression in Pulmonary Hypertensive Rats. Upregulations of PY-STAT3, cyclin D1 and cyclin D3 protein levels were reported in the lungs of Cav-1^{(-/-)} and Cav-2^{(-/-)} mice¹¹. Hyper-activation of the pulmonary STAT3 signaling cascade was also reported in the MI and MCT rat models of PH^{11;13}. An upregulation of both cyclin D1 and cyclin D3 expressions was also observed in the lungs of rats subjected to MI-induced PH¹¹. The present results confirm the hyper-activation of the pulmonary STAT3 signaling cascade and further show marked increases in cyclin D1 and cyclin D3 protein levels in the lungs of MCT and MCT+AP rats. The down-modulation of caveolin proteins may thus represent an initiating mechanism leading to the activation of the STAT3/cyclins pathway and, ultimately, to the development of PH. Accordingly, the present results demonstrate that administration of AP-Cav to MCT rats is sufficient to restore normal levels of pulmonary PY-STAT3, cyclin D1 and cyclin D3. The initiating role of Cav-1 is further supported by the observations of Mathew et al. (2004) which demonstrated that although pulmonary Cav-1 expression decreased as early as 48 hours following the MCT injection, increases in PY-STAT3 were only perceptible at 1 week post-MCT¹³.

Effects of a Cav-1-Derived Peptide on MCT-induced PH and RV Hypertrophy. Cav-1 is well known to interact with many signaling molecules through its caveolin-scaffolding domain (CSD, residues 82-101). Indeed, the CSD recognizes and binds a specific motif within many known proteins such as eNOS, G-alpha subunits, PKC and extracellular signal-regulated kinase-1/2 (ERK1/2)²1²⁶. Interestingly, Cav-1 appears to negatively regulate many of these signaling proteins²⁴⁻²⁶. For instance, a peptide corresponding to the Cav-1 scaffolding domain was previously shown to inhibit the in vitro activity of ERK1/2, and eNOS^{24;26}. Importantly, the generation of caveolin-deficient mice also supports the Cav-1-mediated negative regulation of many proteins, such as eNOS, ERK1/2, cyclins and STAT3^{9;11;27;28}. For instance, Cav-1^{(-/-)} mice display reduced vascular tone and microvascular hyper-permeability secondary to eNOS hyper-activation^{9;27}. Hearts of Cav-1^{(-/-)} mice further display increased ERK1/2 phosphorylation²⁸. As mentioned above, the lungs of Cav-1^{(-/-)} mice also show hyper-activation of the STAT3 signaling cascade, as well as the upregulation of cyclin D1 and cyclin D3 protein levels¹¹. Interestingly, in vivo administration of Cav-1 scaffolding domain peptide was shown to reduce microvascular hyperpermeability, inflammation, and tumor progression in mice^{17;18}. Moreover, perfusion of a Cav-1 peptide was also shown to exert cardioprotective effects in myocardial ischemia-reperfusion experiments by reducing polymorphonuclear neutrophil adherence and infiltration¹⁹. However, whether in vivo administration of such a Cav-1 scaffolding domain peptide could complement the decreased expression of endogenous Cav-1 and prevent the development of PH remained unknown.

The present results show that administration of a cell-permeable Cav-1 peptide to MCT rats prevents increases in pulmonary arteries percent medial wall thickness, RV systolic pressures, and RV/LV+Septum weight ratio. Mechanistically, the present studies show that administration of AP-Cav to MCT rats prevents the reduction of Cav-1 and Cav-2 protein levels, as well as the increases of pulmonary phospho-STAT3, and cyclins protein levels. The reduction of RV systolic pressures observed in MCT+AP-Cav rats could be ascribed, at least in part, to the reduction of pulmonary artery medial hypertrophy. Accordingly, the present studies show that administration of AP-Cav to Control rats has no effects on any of the hemodynamic variables. These results are consistent with previous reports which demonstrated that in vivo delivery of AP-Cav to mice had no effect on the systemic blood pressure, blood flow, and heart rate^{17;18}. Inhibition of the mitogenic STAT3/cyclins pathway observed in the lungs of MCT+AP-Cav rats also supports an essential role for AP-Cav treatment in the prevention of pulmonary artery medial hypertrophy development.

The functional role of caveolin proteins in vascular remodeling also supports the effects of AP-Cav administration on the reduction of pulmonary artery medial hypertrophy. Indeed, reductions in Cav-1 and Cav-2 protein expression were previously shown in the in vitro model of serum-induced vascular smooth muscle cells proliferation²⁹. A decrease of Cav-1 expression was also observed in proliferating smooth muscle cells isolated from human atherosclerotic arteries³⁰. Interestingly, administration of a Cav-1-derived peptide to cultured rat vascular smooth muscle cells was shown to inhibit histamine- and norepinephrine-induced increases in intracellular calcium concentrations through inhibition of phospholipase-C and mitogen-activated protein kinase activation³¹. Importantly, the generation of Cav-1^{(-/-)} mice supports the key regulatory roles of caveolin proteins in smooth muscle cell proliferation and vascular remodeling³². Indeed, cultured aortic smooth muscle cells derived from Cav-1^{(-/-)} mice display increases in proliferation and migration rates, as well as upregulation of phospho-ERK1/2, cyclin D1 and the proliferating cell nuclear antigen protein levels³². Collectively, these reports, as well as the present results, indicate that administration of a cell-permeable Cav-1 peptide might initially prevent the development of pulmonary artery medial hypertrophy which could, consequently, prevent the increases in pulmonary artery pressures and, ultimately, the development of RV hypertrophy. Accordingly, MCT injection has previously been shown to initially stimulate the appearance of muscle in normally non-muscular arterioles, to increase the percent medial wall thickness and to reduce the lumen diameter which, ultimately, results in rises in the pulmonary vascular resistances and pulmonary artery pressures³³.

However, a direct effect of AP-Cav on the RV hypertrophy itself cannot be ruled out. Indeed, decreased expression of caveolin proteins has been documented in the hypertrophic hearts of both spontaneously hypertensive rats and perinephritic hypertensive dogs^{34;35}. Accordingly, both Cav-1^{(-/-)} and Cav-3^{(-/-)} mice were shown to develop RV and LV hypertrophy^{28;36}. Since Cav-1 is not normally expressed in the cardiomyocytes themselves, the development of ventricular and individual cardiomyocyte hypertrophy observed in Cav-1^{(-/-)} mice is most likely to be attributed to the release of autocrine and paracrine factors, such as endothelin-1 and the transforming growth factor-β1. Administration of AP-Cav could thus possibly affect such autocrine/paracrine mechanisms and alter the development of cardiac hypertrophy.

### REFERENCES

1. Scherer P E, Lewis R Y, Volonte D, Engelman J A, Galbiati F, Couet J, Kohtz D S, van Donselaar E, Peters P. Lisanti M P. Cell-type and tissue-specific expression of caveolin-2. Caveolins 1 and 2 co-localize and form a stable hetero-oligomeric complex in vivo. J Biol Chem. 1997; 272:29337-29346.
2. Tang Z, Scherer P E, Okamoto T, Song K, Chu C, Kohtz D S, Nishimoto I, Lodish H F, Lisanti M P. Molecular cloning of caveolin-3, a novel member of the caveolin gene family expressed predominantly in muscle. J Biol Chem. 1996; 271:2255-2261.
3. Song K S, Li S, Okamoto T, Quilliam L A, Sargiacomo M, Lisanti M P. Co-purification and direct interaction of Ras with caveolin, an integral membrane protein of caveolae microdomains. Detergent-free purification of caveolae microdomains. J Biol Chem. 1996; 271:9690-9697.
4. Lisanti M P, Scherer P E, Tang Z L, Sargiacomo M. Caveolae, caveolin and caveolin-rich membrane domains: a signalling hypothesis. Trends Cell Biol. 1994; 4:231-235.
5. Okamoto T, Schlegel A, Scherer P E, Lisanti M P. Caveolins, a family of scaffolding proteins for organizing “preassembled signaling complexes” at the plasma membrane. J Biol Chem. 1998; 273:5419-5422.
6. Smart E J, Graf G A, McNiven M A, Sessa W C, Engelman J A, Scherer P E, Okamoto T, Lisanti M P. Caveolins, liquid-ordered domains, and signal transduction. Mol Cell Biol. 1999; 19:7289-7304.
7. Rothberg K G, Heuser J E, Donzell W C, Ying Y S, Glenney J R, Anderson R G. Caveolin, a protein component of caveolae membrane coats. Cell. 1992; 68:673-682.
8. Glenney J R, Jr., Soppet D. Sequence and expression of caveolin, a protein component of caveolae plasma membrane domains phosphorylated on tyrosine in Rous sarcoma virus-transformed fibroblasts. Proc Natl Acad Sci USA. 1992; 89:10517-10521.
9. Razani B, Engelman J A, Wang X B, Schubert W, Zhang X L, Marks C B, Macaluso F, Russell R G, Li M, Pestell R G, Di Vizio D, Hou H, Jr., Kneitz B, Lagaud G, Christ G J, Edelmann W, Lisanti M P. Caveolin-1 null mice are viable but show evidence of hyperproliferative and vascular abnormalities. J Biol Chem. 2001; 276:38121-38138.
10. Razani B, Wang X B, Engelman J A, Battista M, Lagaud G, Zhang X L, Kneitz B, Hou H, Jr., Christ G J, Edelmann W, Lisanti M P. Caveolin-2-deficient mice show evidence of severe pulmonary dysfunction without disruption of caveolae. Mol Cell Biol. 2002; 22:2329-2344.
11. Jasmin J F, Mercier I, Hnasko R, Cheung M W, Tanowitz H B, Dupuis J, Lisanti M P. Lung remodeling and pulmonary hypertension after myocardial infarction: pathogenic role of reduced caveolin expression. Cardiovasc Res. 2004; 63:747-755.
12. Zhao Y Y, Liu Y, Stan R V, Fan L, Gu Y, Dalton N, Chu P H, Peterson K, Ross J, Jr., Chien K R. Defects in caveolin-1 cause dilated cardiomyopathy and pulmonary hypertension in knockout mice. Proc Natl Acad Sci USA. 2002; 99:11375-11380.
13. Mathew R, Huang J, Shah M, Patel K, Gewitz M, Sehgal P B. Disruption of endothelial-cell caveolin-1alpha/raft scaffolding during development of monocrotaline-induced pulmonary hypertension. Circulation. 2004; 110:1499-1506.
14. Achcar R O, Demura Y, Rai P R, Taraseviciene-Stewart L, Kasper M, Voelkel N F, Cool C D. Loss of caveolin and heme oxygenase expression in severe pulmonary hypertension. Chest. 2006; 129:696-705.
15. Derossi D, Calvet S, Trembleau A, Brunissen A, Chassaing G, Prochiantz A. Cell internalization of the third helix of the Antennapedia homeodomain is receptor-independent. J Biol Chem. 1996; 271:18188-18193.
16. Derossi D, Joliot A H, Chassaing G, Prochiantz A. The third helix of the Antennapedia homeodomain translocates through biological membranes. J Biol Chem. 1994; 269:10444-10450.
17. Gratton J P, Lin M I, Yu J, Weiss E D, Jiang Z L, Fairchild T A, Iwakiri Y, Groszmann R. Claffey K P, Cheng Y C, Sessa W C. Selective inhibition of tumor microvascular permeability by cavtratin blocks tumor progression in mice. Cancer Cell. 2003; 4:31-39.
18. Bucci M, Gratton J P, Rudic R D, Acevedo L, Roviezzo F, Cirino G, Sessa W C. In vivo delivery of the caveolin-1 scaffolding domain inhibits nitric oxide synthesis and reduces inflammation. Nat Med. 2000; 6:1362-1367.
19. Young L H, Ikeda Y, Lefer A M. Caveolin-1 peptide exerts cardioprotective effects in myocardial ischemia-reperfusion via nitric oxide mechanism. Am J Physiol Heart Circ Physiol. 2001; 280:H2489-H2495.
20. Prie S, Leung T K, Ryan J W, Dupuis J. The orally active ET_{A} receptor antagonist (+)-(S)-2-(4,6-dimethoxy-pyrimidin-2-yloxy)-3-metoxy-3,3-diphenyl-propionic acid (LU 135252) prevents the development of pulmonary hypertension and endothelial metabolic dysfunction in monocrotaline-treated rats. J Pharmacol Exp Ther. 1997; 282:1312-1318.
21. Geraci M W, Moore M, Gesell T, Yeager M E, Alger L, Golpon H, Gao B, Loyd J E, Tuder R M, Voelkel N F. Gene expression patterns in the lungs of patients with primary pulmonary hypertension: a gene microarray analysis. Circ Res. 2001; 88:555-562.
22. Tyler R C, Muramatsu M, Abman S H, Stelzner T J, Rodman D M, Bloch K D, McMurtry I F. Variable expression of endothelial NO synthase in three forms of rat pulmonary hypertension. Am J Physiol. 1999; 276:L297-L303.
23. Hironaka E, Hongo M, Sakai A, Mawatari E, Terasawa F, Okumura N, Yamazaki A, Ushiyama Y, Yazaki Y, Kinoshita O, Serotonin receptor antagonist inhibits monocrotaline-induced pulmonary hypertension and prolongs survival in rats. Cardiovasc Res. 2003; 60:692-699.
24. Garcia-Cardena G, Martasek P, Masters B S, Skidd P M, Couet J, Li S, Lisanti M P, Sessa W C. Dissecting the interaction between nitric oxide synthase (NOS) and caveolin. Functional significance of the nos caveolin binding domain in vivo. J Biol Chem. 1997; 272:25437-25440.
25. Couet J, Li S, Okamoto T, Ikezu T, Lisanti M P. Identification of peptide and protein ligands for the caveolin-scaffolding domain. Implications for the interaction of caveolin with caveolae-associated proteins. J Biol Chem. 1997; 272:6525-6533.
26. Engelman J A, Chu C, Lin A, Jo H, Ikezu T, Okamoto T, Kohtz D S, Lisanti M P. Caveolin-mediated regulation of signaling along the p42/44 MAP kinase cascade in vivo. A role for the caveolin-scaffolding domain. FEBS Lett. 1998; 428:205-211.
27. Schubert W, Frank P G, Woodman S E, Hyogo H, Cohen D E, Chow C W, Lisanti M P. Microvascular hyperpermeability in caveolin-1 (−/−) knock-out mice. Treatment with a specific nitric-oxide synthase inhibitor, L-NAME, restores normal microvascular permeability in Cav-1 null mice. J Biol Chem. 2002; 277:40091-40098.
28. Cohen A W, Park D S, Woodman S E, Williams T M, Chandra M, Shirani J, Pereira dS, Kitsis R N, Russell R G, Weiss L M, Tang B, Jelicks L A, Factor S M, Shtutin V, Tanowitz H B, Lisanti M P. Caveolin-1 null mice develop cardiac hypertrophy with hyperactivation of p42/44 MAP kinase in cardiac fibroblasts. Am J Physiol Cell Physiol. 2003; 284:C457-C474.
29. Peterson T E, Kleppe L S, Caplice N M, Pan S, Mueske C S, Simari R D. The regulation of caveolin expression and localization by serum and heparin in vascular smooth muscle cells. Biochem Biophys Res Commun. 1999; 265:722-727.
30. Batetta B, Mulas M F, Petruzzo P, Putzolu M, Bonatesta R R, Sanna F, Cappai A, Brotzu G, Dessi S. Opposite pattern of MDR1 and caveolin-1 gene expression in human atherosclerotic lesions and proliferating human smooth muscle cells. Cell Mol Life Sci. 2001; 58:1113-1120.
31. Ocharan E, Asbun J, Calzada C, Mendez E, Nunez M, Medina R, Suarez G, Meaney E, Ceballos G. Caveolin scaffolding peptide-1 interferes with norepinephrine-induced PLC-beta activation in cultured rat vascular smooth muscle cells. J Cardiovasc Pharmacol. 2005; 46:615-621.
32. Hassan G S, Williams T M, Frank P G, Lisanti M P. Caveolin-1 Deficient (−/−) Aortic Smooth Muscle Cells Show Cell Autonomous Abnormalities in Proliferation, Migration, and Endothelin-based Signal Transduction. Am J Physiol Heart Circ Physiol. 2006.
33. Meyrick B, Gamble W, Reid L. Development of Crotalaria pulmonary hypertension: hemodynamic and structural study. Am J Physiol Heart Circ Physiol. 1980; 239:H692-H702.
34. Piech A, Massart P E, Dessy C, Feron O, Havaux X, Morel N, Vanoverschelde J L, Donckier J, Balligand J L. Decreased expression of myocardial eNOS and caveolin in dogs with hypertrophic cardiomyopathy. Am J Physiol Heart Circ Physiol. 2002; 282:H219-H231.
35. Piech A, Dessy C, Havaux X, Feron O, Balligand J L. Differential regulation of nitric oxide synthases and their allosteric regulators in heart and vessels of hypertensive rats. Cardiovasc Res. 2003; 57:456-467.
36. Woodman S E, Park D S, Cohen A W, Cheung M W, Chandra M, Shirani J, Tang B, Jelicks L A, Kitsis R N, Christ G J, Factor S M, Tanowitz H B, Lisanti M P. Caveolin-3 knock-out mice develop a progressive cardiomyopathy and show hyperactivation of the p42/44 MAPK cascade. J Biol Chem. 2002; 277:38988-38997.
37. Patel H H, Zhang S, Murray F, Suda R Y, Head B P, Yokoyama U, Swaney J S, Niesman I R, Schermuly R T, Pullamsetti S S, Thistlethwaite P A, Miyanohara A, Farquhar M G, Yuan J X, Insel P A. Increased smooth muscle cell expression of caveolin-1 and caveolae contribute to the pathophysiology of idiopathic pulmonary arterial hypertension. FASEB J. 2007 Apr. 30; [Epub ahead of print].
38. Vives, E., Brodin, P., and Lebleu, B. 1997. A truncated HIV-1 Tat protein basic domain rapidly translocates through the plasma membrane and accumulates in the cell nucleus. J. Biol. Chem. 272: 16010-16017.
39. Suzuki, T., Futaki, S., Niwa, M., Tanaka, S., Ueda, K., and Sugiura, Y. 2002. Possible existence of common internalization mechanisms among arginine-rich peptides. J. Biol. Chem. 277: 2437-2443.
40. Coeytaux, E., Coulaud, D., Le Cam, E., Danos, O., and Kichler, A. 2003. The cationic amphipathic {alpha}-helix of HIV-1 viral protein R (Vpr) binds to nucleic acids, permeablizes membranes, and efficiently transfects cells. J. Biol. Chem. 278: 18110-18116.
41. Schwarze, S. R., Ho, A., Vocero-Akbani, A., and Dowdy, S. F. 1999. In vivo protein transduction: Delivery of a biologically active protein into a mouse. Science 285: 1569-1572.
42. Rothbard, J. B., Kreider, E., VanDeusen, C. L., Wright, L., Wylie, B. L., and Wender, P. A. 2002. Arginine-rich molecular transporters for drug delivery: Role of backbone spacing in cellular uptake. J. Med. Chem. 45: 3612-3618.
43. Mitchell, D. J., Kim, D. T., Steinman, L., Fathman, C. G., and Rothbard, J. B. 2000. Polyarginine enters cells more efficiently than other polycationic homopolymers. J. Pept. Res. 56: 318-325.
44. Fuchs S M, Raines R T. Polyarginine as a multifunctional fusion tag. Protein Science 14: 1538-1544, 2005.
45. U.S. Pat. No. 6,335,320, issued Jan. 1, 2002, Gabbiani et al. Method of treating fibrotic conditions.

1. A method of preventing and/or treating pulmonary hypertension and/or right ventricular hypertrophy comprising administering to a patient an amount of a caveolin peptide coupled to a cell permeating compound effective to prevent and/or treat pulmonary hypertension and/or right ventricular hypertrophy in a patient.

2. The method of claim 1, wherein pulmonary hypertension is prevented.

3. The method of claim 1, wherein pulmonary hypertension is treated.

4. The method of claim 1, wherein right ventricular hypertrophy is prevented.

5. The method of claim 1, wherein right ventricular hypertrophy is treated.

6. The method of claim 1, wherein the pulmonary hypertension is primary pulmonary hypertension.

7. The method of claim 1, wherein the pulmonary hypertension is secondary pulmonary hypertension.

8. The method of claim 1, wherein the caveolin peptide coupled to the cell permeating compound is administered systemically.

9. The method of claim 1, wherein the caveolin peptide coupled to a cell permeating compound is administered by inhalation.

10. The method of claim 1, wherein the order of the caveolin peptide coupled to the cell permeating compound is first cell permeating compound then caveolin peptide.

11. The method of claim 1, wherein the order of the caveolin peptide coupled to the cell permeating compound is first caveolin peptide then cell permeating compound.

12. The method of claim 1, wherein the caveolin peptide is one or more of:

| | |
|---|---|
| DGIWKASFTTFTVTKYWFYR (Cav-1); | (SEQ ID NO:7) |
| DKVWICSHALFEISKYVMYK (Cav-2); | (SEQ ID NO:8) |
| DGVWRVSYTTFTVSKYWCYR (Cav-3); | (SEQ ID NO:9) |

or a peptide which both has at least 80% identity with Cav-1, Cav-2 or Cav-3 and inhibits a GTPase, a NOS and a kinase.

13. The method of claim 1, wherein the caveolin peptide is DGIWKASFTTFTVTKYWFYR (Cav-1) (SEQ ID NO:7).

14. The method of claim 1, wherein the cell permeating compound is a liposome, a lipid, a fatty acid, or a peptide.

15. The method of claim 1, wherein the caveolin peptide is coupled to a cell permeating peptide.

16. The method of claim 15, wherein the cell permeating peptide is one or more of:
(SEQ ID NO:1)
RQPKIWFPNRRKPWKK;
(SEQ ID NO:2)
RQIKIWFQNRRMKWKK (penetratin);
(SEQ ID NO:3)
a polypeptide consisting of 6-15 arginines;
(SEQ ID NO:4)
GYGRKKRRQRRRG (TAT);
(SEQ ID NO:5)
YGRKKRRQRRR (TAT);
(SEQ ID NO:6)
GRKKRRQRRRPPQ (TAT);
or a cell permeating peptide having at least 80% identity with any of said cell permeating peptides.

17. The method of claim 16, wherein the cell permeating peptide having at least 80% identity with any of said cell permeating peptides comprises at least three arginines.

18. The method of claim 15, wherein the cell permeating peptide is RQPKIWFPNRRKPWKK (SEQ ID NO:1).

19. The method of claim 15, wherein the peptide consisting of the cell permeating peptide coupled to the caveolin peptide is:
(SEQ ID NO:10)
RQPKIWFPNRRKPWKK-DGIWKASFTTFTVTKYWFYR
or
(SEQ ID NO:11)
DGIWKASFTTFTVTKYWFYR-RQPKIWFPNRRKPWKK.

20. The method of claim 15, wherein the peptide consisting of the cell permeating peptide coupled to the caveolin peptide is:
(SEQ ID NO:10)
RQPKIWFPNRRKPWKK-DGIWKASFTTFTVTKYWFYR.

21. The method of claim 1, wherein the caveolin peptide is coupled to a cell permeating fatty acid.

22. The method of claim 21, wherein the cell permeating fatty acid is palmitate or myristate.

23. A cell permeating peptide having the sequence RQPKIWFPNRRKPWKK (SEQ ID NO:1).

24. The cell permeating peptide of claim 23, wherein the cell permeating peptide is coupled to a caveolin peptide.

25. The cell permeating peptide of claim 24, wherein the order of the caveolin peptide coupled to the cell permeating peptide is first cell permeating peptide then caveolin peptide.

26. The cell permeating peptide of claim 24; wherein the order of the caveolin peptide coupled to the cell permeating peptide is first caveolin peptide then cell permeating peptide.

27. The cell permeating peptide of claim 24, wherein the caveolin peptide is one or more of:

| | |
|---|---|
| DGIWKASFTTFTVTKYWFYR (Cav-1); | (SEQ ID NO:7) |
| DKVWICSHALFEISKYVMYK (Cav-2); | (SEQ ID NO:8) |
| DGVWRVSYTTFTVSKYWCYR (Cav-3); | (SEQ ID NO:9) |

or a peptide which both has at least 80% identity with Cav-1, Cav-2 or Cav-3 and inhibits a GTPase, a NOS and a kinase.

28. The cell permeating peptide of claim 24, wherein the caveolin peptide is DGIWKASFTTFTVTKYWFYR (Cav-1) (SEQ ID NO:7).

29. The cell permeating peptide coupled to the caveolin peptide of claim 24 comprising the peptide sequence:
(SEQ ID NO:10)
RQPKIWFPNRRKPWKK-DGIWKASFTTFTVTKYWFYR
or
(SEQ ID NO:11)
DGIWKASFTTFTVTKYWFYR-RQPKIWFPNRRKPWKK.

30. The cell permeating peptide coupled to the caveolin peptide of claim 24 comprising the peptide sequence:
(SEQ ID NO:10)
RQPKIWFPNRRKPWKK-DGIWKASFTTFTVTKYWFYR.


SEQUENCE LISTING (11 sequences)

SEQ ID NO: 1 - protein, 16 aa, Artificial Sequence
  cell permeating peptide
RQPKIWFPNRRKPWKK

SEQ ID NO: 2 - protein, 16 aa, Drosophilia
RQIKIWFQNRRMKWKK

SEQ ID NO: 3 - protein, 15 aa, Artificial Seqence
  The number of Arg could be anywhere between 6-15, a cell permeating peptide
RRRRRRRRRRRRRRR

SEQ ID NO: 4 - protein, 13 aa, Artificial Sequence
  a TAT sequence, TAT is from the HIV virus, a cell permeating peptide
GYGRKKRRQRRRG

SEQ ID NO: 5 - protein, 11 aa, Artificial Sequence
  a TAT sequence, TAT is from the HIV virus, a cell permeating peptide
YGRKKRRQRRR

SEQ ID NO: 6 - protein, 13 aa, Artificial Sequence
  a TAT sequence, TAT is from the HIV virus, a cell permeating peptide
GRKKRRQRRRPPQ

SEQ ID NO: 7 - protein, 20 aa, Human
DGIWKASFTTFTVTKYWFYR

SEQ ID NO: 8 - protein, 20 aa, Human
DKVWICSHALFEISKYVMYK

SEQ ID NO: 9 - protein, 20 aa, Human
DGVWRVSYTTFTVSKYWCYR

SEQ ID NO: 10 - protein, 36 aa, artificial sequence
  cell permeating peptide coupled to caveolin peptide
RQPKIWFPNRRKPWKKDGIWKASFTTFTVTKYWFYR

SEQ ID NO: 11 - protein, 36 aa, artificial sequence
  cell permeating peptide coupled to caveolin peptide
DGIWKASFTTFTVTKYWFYRRQPKIWFPNRRKPWKK